(19) United States
(12) Patent Application Publication
(10) Pub. No.: US 2006/0246431 A1
(43) Pub. Date: Nov. 2, 2006

(54) Test apparatus

(76) Inventor: Wamadiva Balachandran, Middlesex (GB)

Correspondence Address: DeWitt Ross & Stevens;Intellectual Porperty Department, US Bank Building, Suite 401 8000 Excelsior Drive, Madison, WI, 53717-1914, US

(21) Appl. No.: 10/499,831

(22) PCT Filed: Dec. 9, 2002
(86) PCT No.: PCT/GB02/05553

§ 371 Date: Apr. 6, 2005

(30) Foreign Application Priority Data
Dec. 7, 2001 (GB) 0129374.5

(51) Int. Cl. C12Q 1/68; C12M 1/34

(52) U.S. Cl. 435/6; 435/287.2

(57) ABSTRACT

Test apparatus includes a sample receiver operable to receive a sample to be tested, a sample manipulator operable to manipulate a received sample, a reagent receiver operable to add to the received sample a reagent, and a result indicator operable to indicate whether the received sample contains a substance reactable with the reagent. Two or more of the elements of the apparatus are provided on a common substrate. The apparatus is designed to test small size samples, for example a drop of blood or less, typically a sample of the order of 5 μl. It includes a manipulator able to manipulate a small size sample and an electrical field generator operable to move a sample under test through the apparatus. The elements of the apparatus are provided on a common substrate and the apparatus includes an electrical manipulator for manipulating a sample, at least one micropump pumping and mixing sample with a reagent and an indicator for indicating whether the reagent has indicated the existence of a specified component within the sample under test. The apparatus can be in the form a kit which provides a sterile, disposable, consumable laboratory-on-a-chip device.

The present invention relates to test apparatus which could be described in some embodiments as a laboratory on a chip.

There is a great variety of situations in which it is necessary to test a substance, for medical, pathological or other reasons, where there would be real advantages in obtaining the test results quickly. However, in most instances samples to be tested must be sent to a laboratory for analysis, which is time consuming, also leading to possibility that the results are not obtained in time.

The present invention seeks to provide an improved test system.

According to an aspect of the present invention, there is provided test apparatus including sample receiving means operable to receive a sample to be tested, sample manipulating means operable to manipulate a received sample, reagent receiving means operable to add to the received sample a reagent and result indicating means operable to indicate whether the received sample contains a substance reactable with the reagent; wherein two or more of the elements of the apparatus are provided on a common substrate.

Preferably the test apparatus is designed to test small size samples, for example a drop of blood or less. The preferred embodiment described below is designed to test a sample of the order of 5 μl. For this purpose, the preferred embodiment provides manipulating means able to manipulate a small size sample.

Advantageously, the manipulating means includes electrical field generating means operable to move a sample under test through the apparatus.

In the preferred embodiment there is in addition or alternatively provided a micropump operable to pump and mix sample substantially simultaneously. The pump is preferably a piezoelectric pump or could be any other form of micropump.

In the preferred embodiment, the elements of the apparatus are all provided on a single substrate or chip. In practice, it is envisaged that the chip would be of a reasonably small size to make it easy to handle by a person and can therefore be kept at the point of use, for example at a doctor's surgery and at a food processing plant and the like.

Advantageously, the means for receiving the reagent includes a reagent housing operable to store one or a selected number of reagents. In this manner, the apparatus can be set up ready to carry out an analysis upon the introduction of a sample into the apparatus. For example, the apparatus could be designed to test for a particular DNA/RNA characteristic, for a particular pathogen or other component within a sample to be analysed. This can make the carrying out of analysis on a sample much easier by reducing the chances or error and provides a system in which testing can be carried out very rapidly, for example while a patient is waiting at a doctor's surgery or at a food processing line.

In a preferred embodiment, the electrical manipulating means provides a plurality of electrodes arranged in an array and powered to create via an electrical field a dielectrophoretic force, dependent upon particle sizes within the sample and their dielectric properties. The electrodes preferably include at least a portion angled into the intended direction motion of a sample under test within the apparatus. In the preferred embodiment, each electrode includes an end having a surface parallel or substantially parallel to said intended direction of motion. The intended direction of motion is preferably delimited by a conduit within the apparatus. The conduit could be straight, curved or angled as required or desired.

In the preferred embodiment, the elements of the apparatus are provided on a common substrate and the apparatus includes electrical manipulating means for manipulating a sample, at least one micropump pumping and mixing sample with a reagent and indicator means for indicating whether the reagent has indicated the existence of a specified component within the sample under test. This can be a very convenient apparatus which can be manufactured at relatively low cost which can therefore be used on a one-off basis and at the point of use.

The electrode array of the preferred embodiment, it has been found, is able to move very small particle sizes down to nanoparticles, making the apparatus able to test very small sample quantities.

The preferred embodiment can thus provide a rapid and cost effective diagnostic method and kit to perform the increasing numbers of tests required for clinical diagnosis (such as cancer detection), based on DNA and RNA. The kit would provide a sterile, disposable, consumable laboratory-on-a-chip device able to perform large numbers of diagnostics in a simple, user friendly format. It can have the added benefit of being cost effective and part of a self-contained unit and so suitable for “point-of-care” diagnostics (for example at a doctor's surgery). Such a method and kit have many applications and can be applied to food supplies and so identify pathogens, the detection of variations and mutations in genes and evolutionary genetics.

In one implementation, there is provided a tested and robust DNA laboratory-on-a-chip and software controlled instrument for the rapid and multiplex detection of a number of DNA/RNA fragments in the same sample. The system can enable any distinguishable DNA/RNA sequences to be detected and so will spawn a wide range of assays for food pathogens and subsequently a wider range of human diagnostics.

The apparatus is designed to integrate many existing MEMs technologies, including micro-fluidics, polymerise chain reaction (PCR) techniques.

The preferred embodiment utilises the phenomenon of dielectrophoretic force for DNA/RNA fragment population manipulation, characterisation and entrapment. It has been found that dielectrophoresis, which occurs when a polarizable particle is subjected to the application of a non-uniform electric field, when controlled precisely allows accurate manoeuvring and classification of nanometre sized biological particles. The preferred device can sort DNA/RNA fragments of the same size and/or dielectric properties into holding locations, where their presence, size or dielectric properties are detected.

The integration of these known technologies and their application to detection of specific populations of DNA/RNA fragments in highly complex samples in a closed system can provide substantial advantages and independence from human error. For example, blood samples from a patient suspected of having a disease or a food sample suspected of containing pathogens, can be detected reliably, specifically and quantifiably.

The preferred device exploits flexible software control of the functions within the laboratory-on-a-chip, together with computer data collection and processing. This method allows many tests to be performed on the same sample, within a short time, by relatively unskilled staff.

Embodiments of the present invention are described below, by way of example only, with reference to the accompanying drawings, in which:

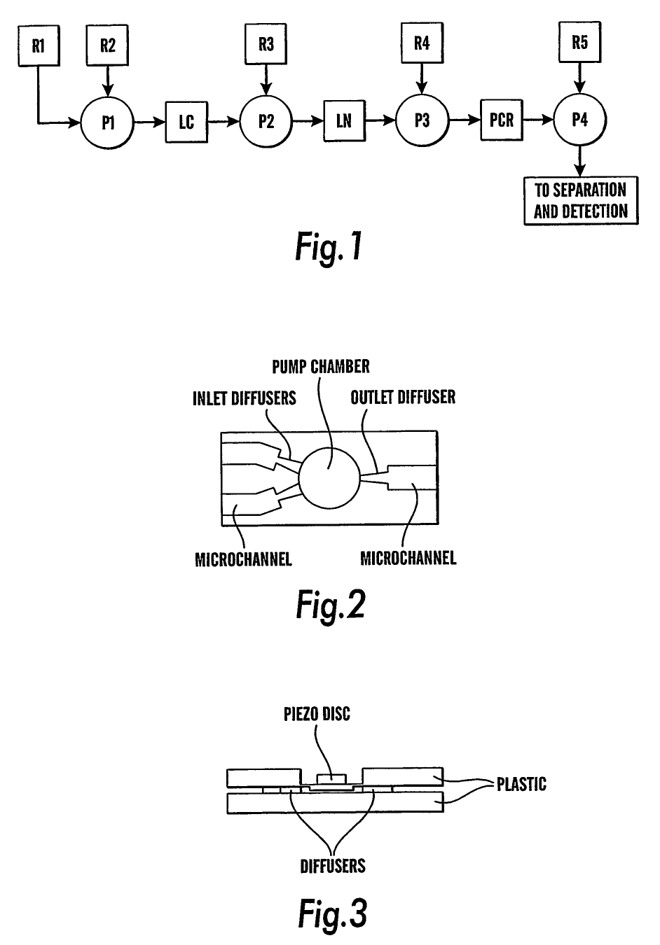

FIG. 1 is a schematic diagram of an embodiment of laboratory on a chip;

FIG. 2 is schematic plan view of an embodiment of micropump;

FIG. 3 is a side elevational view of the micropump of FIG. 2;

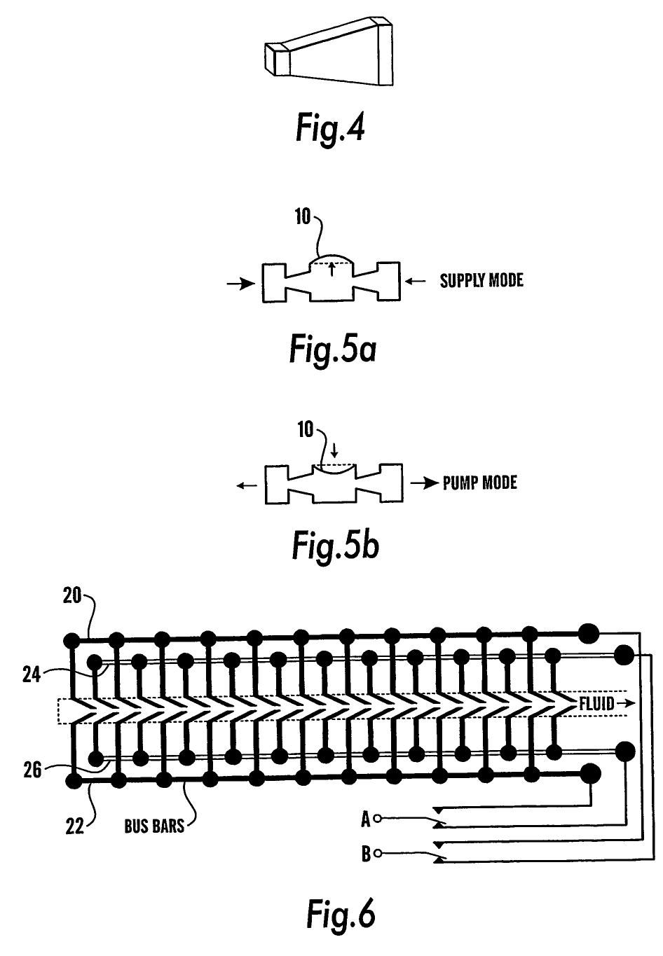

FIG. 4 is a schematic view of an embodiment of diffuser of the micropump of FIGS. 2 and 3;

FIGS. 5a and 5b show the micropump in use;

FIG. 6 shows in plan view the preferred embodiment of electrode array for the apparatus of FIG. 1;

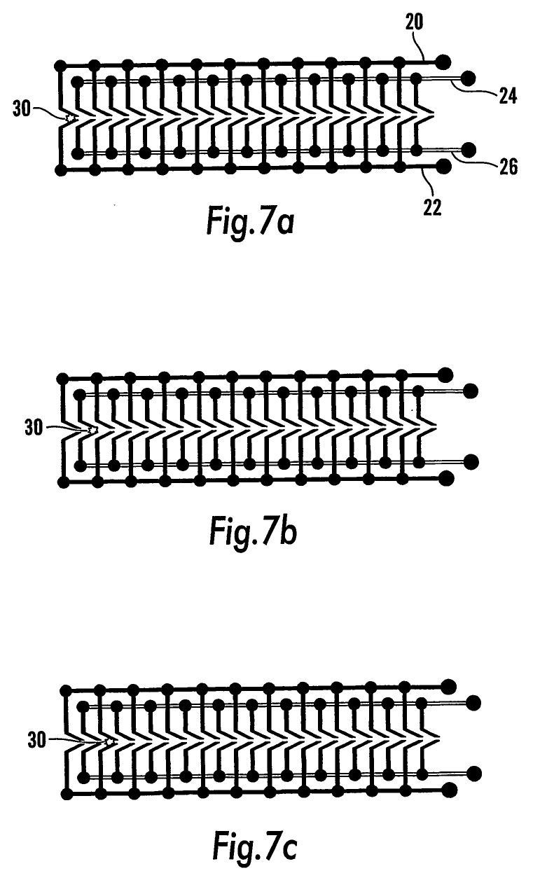

FIGS. 7a to 7c show the motion of a microparticle through the electrode array of FIG. 6;

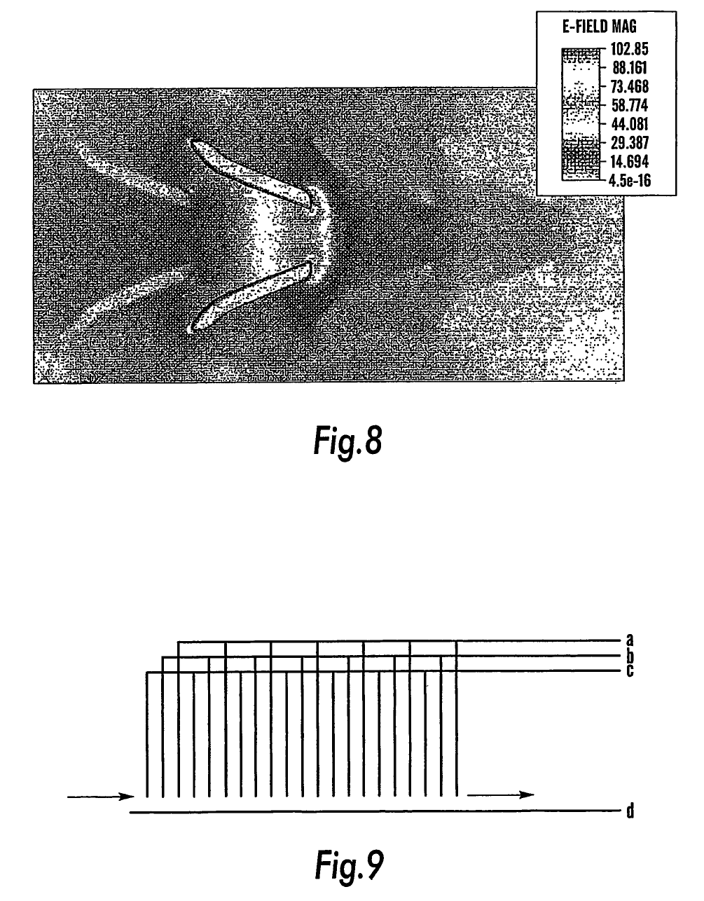

FIG. 8 shows a finite element model of the field magnitude surrounding the electrode array and embodiment; and

FIG. 9 shows a simple electrode arrangement suitable for positive dielectrophoretic movement.

The preferred embodiment can be described as a laboratory on a chip, being a combination of the various elements required to manipulate a sample to be tested, to test the sample and to provide an indication of the test results. For example, when testing a sample of blood in a doctor's laboratory, the apparatus can be configured to receive a sample of blood, manipulate that sample so as to extract from the sample the relevant component to be tested (a disease, DNA strand, for example) to add to the extracted component a reagent, to mix the reagent and extract the component together and to analyse the results of the reaction to determine whether or not the sample includes the component in question.

It is envisaged that this apparatus would be provided all on a common substrate of relatively small dimensions, for example which can be easily carried by a person and possibly in the form of a very small substantially flat article which can be easily packaged and transported. The indicator could be, for example, one or more visual indicators such as lights (LED's or alternative) which may give a simple yes/no indication.

Of course, the device could be designed to give an addition to such a qualitative indication a quantitative indication in cases where it is necessary to know what quantity of a component present in the sample under test.

In operation, a sample of fluid to be investigated is introduced into the device via an input port system (described in further detail below). Within the device there exist sterile controlled surroundings, isolated from external environmental contamination. Micro-fluidic technology is employed in the extraction and purification of a component (for example DNA material) from the sample. In the case of DNA/RNA sampling, standard PCR methodology can be used if necessary (the device can allow for very small particles to be manipulated with accuracy, possibly obviating the need for amplification in some cases). The obtained DNA fragment particle population scan be added to a carrier medium suitable for dielectrophoretic control, provided by novel dielectrophoresis sorting using electrode arrays. The sorting allows the categorisation of individual particle populations. Fragment particle populations of DNA/RNA are then held by dielectrophoretic forces within holding locations. Such dielectrophoretic traps allow rotation of particles, enhancing characterisation technique. Finally, the detection of presence or absence of expected particle populations is recorded within software, testing for the occurrence of specific detectable DNA/RNA strand.

Compared with conventional laboratory analysis methods, very small quantities of test sample and reagent, typically tens of microlitres can be used; this advantage being combined with rapid detection times.

FIG. 1 shows a specific example of such a laboratory on a chip. The example is directed to the extraction and analysis of DNA/RNA fragments. However, the test to be performed is dependent upon a user's wishes.

This embodiment for the microfiuidic part of the chip for DNA separation and detection is based on the chemical protocol for simple alkaline extraction of human genomic DNA for PCR amplification. The protocol consists of the following steps:
1. mixing raw sample (for instance, 5 μl whole blood) with 5 μl of 10 mM NaOH,
2. heating to 95° C. for 1-2 minutes to lyse cells, releasing DNA and denaturing proteins inhibitory to PCR,
3. neutralisation of lysate by mixing with 5 μl of 16 mM tris-HCl (pH=7.5),
4. mixing of neutralised lysate with 8-10 μl of liquid PCR reagents and user selected primers, and
5. thermal cycling for PCR amplification.

All of the volumes stated above may be proportionally decreased regarding to the dimensions of the microchannels and chambers in the final chip design.

FIG. 1 shows an example of on-chip implementation for this method described above.

The principal design elements of the micropumps P1-P4 are described in detail below.

The elements on the chip are as follows. R1 and R2 are reservoirs for sample and NaOH load, respectively. From each of these reservoirs, a microfluidic channel leads to the pump P1. P1 is a valve-less diffuser micropump with a piezoelectric actuation (the principal design being shown below). The role of the pump P1 is twofold. First, it pumps the blood sample and NaOH from the reservoirs to a lysis chamber (LC block on the diagram). Secondly, the pump serves as a mixer, providing the mixing of a sample and NaOH for a better yield of the lysis process. The mixing of fluids in the pump relies on the fact that the flow pattern inside the pump chamber is designed to be vortex-like, due to the diaphragm deflections.

As stated, LC is the chamber where the process of lysis takes place. When loaded into the chamber, the mixture is heated to 95° C. for 1-2 minutes by on-chip platinum heaters (not shown) placed at the bottom or under the bottom of the chamber. The next unit is the micropump P2, which pumps the lysate from the lysis chamber and tris-HCl from the reservoir R3, mixes the two and pumps the mixture towards the neutralised lysate holding chamber. The latter is represented in FIG. 1 by the LN block. The pump P3 pumps the neutralised lysate from the LN chamber and mixes it with PCR reagents and primers, previously loaded into reservoir R4. The PCR mixture is then pumped into the PCR chamber, where 35 cycles of heating and cooling are performed for DNA amplification. On-chip platinum heaters (not shown) are placed at the bottom or under the bottom of the PCR chamber for the mixture heating.

Finally, after the PCR amplification has been completed, the pump R4 pumps the PCR product from the PCR chamber and mixes it with the fluid carrier for dielectrophoretic separation in the subsequent part of the chip. The fluid carried has previously been loaded into the reservoir R5. The mixture is then pumped to the dielectrophoretic part of the chip (described in detail below).

FIGS. 1, 2 and 3 show the preferred design of valveless diffuser micropump with two inlet diffusers for use as the pumps P1, P2, P3 and P4.

A key element in the micropump is the diffuser, which is a flow channel with gradually expanding cross-section. The diffuser can have either rectangular or circular cross-section, but the rectangular one is chosen here because it is of a more compact design. An example of rectangular diffuser is shown in FIG. 4.

The most interesting feature of the diffuser is the difference in its flow resistances in the forward (diffuser) and reverse (nozzle) directions. The net flow resulting from this fact can be recognised from FIGS. 5a and 5b, where the sizes of arrows correspond to the amount of the liquid pumped into the chamber in the supply mode and out of the chamber in the pump mode.

In the supply mode, the diaphragm 10 deflection in the shown direction causes fluid to be pumped into the chamber from both the pump inlet and outlet channels. The fluid coming from the inlet side has to flow through the diffuser to get into the pump chamber, while the fluid coming from the outlet side has to flow through the nozzle. The flow resistance that the fluid encounters coming from the inlet side is smaller (the flow resistance of the diffuser is smaller than that of the nozzle). Therefore the fluid volume introduced to the pump chamber from that side is greater than the one introduced from the outlet side, where the greater flow resistance is encountered.

In the pump mode, the diaphragm 10 deflection causes the fluid to be pumped out of the pump chamber, again towards both inlet and outlet. In this case, the fluid flowing towards the inlet side encounters greater flow resistance and the greater volume is pumped towards the outlet side, where the flow resistance is smaller.

In conclusion, the pump diaphragm 10 deflections cause the net flow of the fluid from the inlet side to the outlet side.

The estimation of the pumping efficiency is based on the diffuser element efficiency ratio as follows. For the diffuser element, the pressure drop in the diffuser and in the nozzle directions can be written as follows.

$$Δ {p}_{diffuser} = {ξ}_{diffuser} * \frac{1}{2} ρ {\hat{u}}_{diffuser}^{2} , \\ Δ {p}_{nozzle} = {ξ}_{nozzle} * \frac{1}{2} p {\hat{u}}_{nozzle}^{2} ,$$

where ξ is the pressure loss coefficient, ρ is the fluid density and û is the mean fluid velocity in the diffuser neck. The diffuser element efficiency ratio is then defined as

$$η = \frac{{ξ}_{nozzle}}{{ξ}_{diffuser}} .$$

The optimum efficiency is achieved when η is maximised. The experimental analysis showed that η is maximised for the angle between the diffuser walls equal to approximately 5°. The diffuser angles in FIGS. 1 and 2 are drawn greater than 5° merely for better representation.

There are certain advantages that make this design of pump suitable for handling fluids in a laboratory-on-a-chip device, of the type taught herein, comparing to other ways of pumping. For example, many of the present micro analytical systems are electro-osmotic and electro-hydrodynamic pumping. In these cases the pumping effect is based on certain properties of the fluid, namely pH and ionic strength, which makes them unsuited for a large number of biological fluids. The pump mechanism presented here is able to handle wide variety of fluids, practically independent on their chemical and physical properties. Another advantage of this pump is the simple valve-less design. The valves in microfluidic devices are susceptible to decrease in performance after some time of operation due to wear and fatigue. Also, if the diaphragm deflection does not produce a pressure difference that is high enough to open the valve when the pump chamber is filled with gas, the chamber must be filled with liquid before successful pumping can occur, which is impractical in many cases. The pump with the valve-less design taught here is self-priming, it is not necessary to fill the chamber with liquid for pumping to be successful. Additionally, when the pumped fluid is used to carry particles, valves are susceptible to clogging.

The flow rate of the fluid pumped with this pump depends upon the frequency of the piezoelectric disc actuator. The system has a resonant frequency, representing the frequency at which the fluid flow is maximised. However, if the pump has two inlets and serves as a mixer of two different types of fluids, an optimum has to be found in the diffuser design and applied frequency to ensure the mixing with the desired ratio.

The pump was also tested against gas bubbles introduced to the fluid in the channel, but they were washed away without any significant impact on pump performance. The shallow pump chamber and a thin diaphragm (both a few tens of micrometres) produced a compression high enough to wash the bubbles away easily.

The pump showed a great capability to handle the fluids with a large concentration of beads or cells. Cells were tested after being pumped and they proved viable, without any ruptures being detected.

The temperature in the pump chamber increases when pump is operating due to the heating of piezoelectric disc. The temperature of the disc increases with the frequency of the applied voltage, but in this case the frequency can be low enough (400-700 Hz) for the temperature increase to be acceptable. The reported increase of the temperature of the pumped water was 1.1° C. (5%) and 2.4° C. (11%) for the frequencies of 1 and 3 kHz, respectively. This is well below the critical temperature for biological samples.

As stated above, the pump design described here has very good performance when pumping the wide variety of fluids, including biological samples. Its simple design makes the pump attractive by means of its integration into a laboratory-on-a-chip device.

In the preferred embodiment, micrometre sized and sub-micrometre sized particles, including biological particles, are conveyed and sorted using an electrode arrangement similar to that shown in FIG. 6.

The preferred electrode array uses a series of pairs of angled electrodes. Alternate electrodes on each side of the fluid channel, designated by horizontal dotted lines, are electrically connected. The electrodes are made from an electrically conductive material, such as gold or platinum, deposited on an inert substrate. Field strengths in the order of 10⁶ V/m are used.

In operation, a liquid carrier medium is used. This occupies the fluid channel between the electrode pairs. An alternating voltage is applied to the outer pair 20, 22 of the four electrode bus bars shown in FIG. 6. Particles 30 exhibiting positive dielectrophoretic characteristics when placed in the carrier fluid, on the left hand side of the array at the start of the electrode arrangement, will be attracted towards the area of high field intensity that exists between the points of the first electrode pair as shown in FIG. 7a.

Tuning the frequency of the applied ac signal allows the application of maximum dielectrophoretic force to an individual particle population type.

Changing the applied ac signal to the inner pair 24, 26 of electrode bus bars creates high field intensity at the tip of the second angled pair of the electrodes, as shown in FIG. 7b. This high field intensity attracts particles from the area of the tips of the first pair of electrodes; no voltage is now being applied to the first electrode pair.

When particles have reached the tip of the second pair of electrodes the applied signal is again switched to the outer pair of bus bars 20, 22 connecting the electrode pairs. The first, third, etc. pairs of electrodes now have the applied ac signal across them. An area of high field intensity at the tip of the third electrode pair now attracts the particles that are in the region of the second electrode pair tips, as shown in FIG. 7b.

Particles are moved along the electrode array by switching the applied ac signal between the outer and inner pair of bus bars, as shown in FIG. 6. The frequency at which the applied signal to A and B in FIG. 6 is switched between the outer and inner bus bar pairs may be altered. This allows the fastest moving particles 30 to be moved between individual high intensity regions with the greatest efficiency, leaving behind slower moving particles, enhancing particle sorting.

An Algor software simulation of the field intensity surrounding a pair of electrodes in the electrode array is shown in FIG. 8.

An alternative electrode arrangement connects every third electrode pair to a common bus bar. The signal is applied sequentially to each pair of electrodes. A simple analogy is that of travelling lights in a display. Positive dielectrophoretic force propels the particles towards regions of high field strength. An example of this switching arrangement is shown in FIG. 9. Point d is one side of an ac supply (for example 5 MHz), voltage is switched sequentially from a to b to c (for example at 1 KHz). A non-uniform field is created at the end of the electrodes attached to the bus lines a, b, c and the electrode d. Particles are drawn along the electrode arrangement by positive dielectrophoretic force.

Using arrays of electrodes having dimensions in the order of a few micrometres enables the production of high fields strengths using low voltages, and also allows the application of high frequency signals, which helps minimise electro hydrodynamic fluid pumping within the carrier medium.

In the preferred embodiment, each electrode of the electrode array has a pointed tip extending into the path which particles take. In the preferred embodiment, the tip of each electrode is provided with a flat surface which is substantially parallel to the conduit over which the electrode extends and in practice is substantially parallel to the movement of particles within the conduit. It has been found that providing such a fiat surface avoids field peaks which could under some circumstances adversely affect the movement of the particles.

1-17. (canceled)

18. A test apparatus including:
a. a sample receiver operable to receive a sample to be tested,
b. a sample manipulator operable to manipulate a received sample,
c. a reagent receiver operable to add to the received sample a reagent, and
d. a result indicator operable to indicate whether the received sample contains a substance reactable with the reagent,
wherein two or more of the elements of the apparatus are provided on a common substrate.

19. A test apparatus according to claim 18 wherein the apparatus is designed to test small size samples having a volume approximately equivalent to or less than the volume of a drop of blood.

20. A test apparatus according to claim 19 wherein the apparatus is designed to test a sample having a volume on the order of 5 μl.

21. A test apparatus according to claim 18 wherein the sample manipulator includes an electrical field generator operable to move a sample under test through the apparatus.

22. A test apparatus according to claim 18 further including a micropump operable to pump and mix sample substantially simultaneously.

23. A test apparatus according to claim 22 wherein the pump is a piezoelectric pump.

24. A test apparatus according to claim 18 wherein the elements of the apparatus are all provided on a single substrate.

25. A test apparatus according to claim 18 wherein the reagent receiver includes a reagent housing operable to store one or more reagents.

26. A test apparatus according to claim 18 wherein the apparatus is designed to test for a particular DNA/RNA characteristic.

27. A test apparatus according to claim 18 further including a plurality of electrodes arranged in an array and powered to create via an electrical field a dielectrophoretic force, dependent upon particle sizes within the sample and their dielectric properties.

28. A test apparatus according to claim 27 wherein the electrodes include at least a portion angled into the intended direction motion of a sample under test within the apparatus.

29. A test apparatus according to claim 28 wherein each electrode includes an end having a surface parallel or substantially parallel to said intended direction of motion.

30. A test apparatus according to claim 29 wherein the intended direction of motion is delimited by a conduit within the apparatus.

31. A test apparatus according to claim 30 wherein the conduit has one of a straight, curved and angled configuration.

32. A test apparatus according to claim 18 wherein:
a. all elements of the apparatus are provided on a common substrate; and
b. the apparatus further includes:
(1) at least one micropump pumping and mixing sample with a reagent, and
(2) an indicator for indicating whether the reagent has indicated the existence of a specified component within the sample under test.

33. A kit including:
a. a test apparatus according to claim 18; and
b. a reagent.

34. A kit according to claim 33 wherein the kit provides a sterile, disposable, consumable laboratory-on-a-chip device.

35. A test apparatus including the following elements arrayed on a common substrate:
a. a sample reservoir wherein a sample to be tested may be received,
b. a reagent reservoir wherein a reagent may be received,
c. one or more pumps actuatable to manipulate the received sample and reagent,
d. a heating chamber wherein mixed sample and reagent are heated; and
e. electrodes chargeable to act on products of the mixture.
